(19) United States
(12) Patent Application Publication
(10) Pub. No.: US 2026/0144744 A1
(43) Pub. Date: May 28, 2026

(54) NASAL SPRAY SOLUTION FOR HANGOVERS AND METHOD FOR ITS PREPARATION

(71) Applicant: Christian Butura, Scottsdale, AZ (US)

(72) Inventor: Christian Butura, Scottsdale, AZ (US)

(21) Appl. No.: 18/957,718

(22) Filed: Nov. 23, 2024

(51) Int. Cl. A61K 9/00; A61K 31/047; A61K 31/198; A61K 31/375; A61K 31/4415; A61K 31/505; A61K 31/714; A61K 33/14; A61K 36/886; A61K 47/02; A61K 47/18

(52) U.S. Cl. CPC A61K 9/0043; A61K 31/047; A61K 31/198; A61K 31/375; A61K 31/4415; A61K 31/505; A61K 31/714; A61K 33/14; A61K 36/886; A61K 47/02; A61K 47/186

(57) ABSTRACT

A nasal spray solution for hangovers and method for its preparation. The nasal spray solution may be used to alleviate headache, fatigue, nausea, redness of the eyes, raised blood pressure, congestion, dizziness and other symptoms resulting from the consumption of alcohol. The main active components of the nasal spray solution are vitamin B12, aloe vera extract, L-glutamine, and potassium chloride.

### TECHNICAL FIELD

This disclosure relates generally to nasal spray. More specifically, the present invention is a nasal spray solution for hangovers and method for its preparation.

### BACKGROUND OF THE INVENTION

Alcohol consumption often results in a hangover, which may include a variety of unpleasant symptoms. Some common hangover symptoms include headache, fatigue, nausea, redness of the eyes, raised blood pressure, congestion, and dizziness. The types of symptoms and the severity of the symptoms may vary from person to person and may also be influenced by the amount of alcohol consumed by that person.

Hangover symptoms may take about 24 hours to resolve. Pain relievers may be used to help alleviate some of the symptoms. However, the pain relievers may take too long to take effect. Some individuals may also feel self-conscious about taking pain relievers in public. Therefore, a need exists for a fast acting and inconspicuous remedy for hangover symptoms.

The present disclosure provides for a nasal spray solution for relieving hangover symptoms. The nasal spray disclosed herein is quickly absorbed, is fast acting, and may be administered discretely because it looks like a typical nasal spray that one might use for allergies. Other benefits and advantages will become clear from the disclosure provided herein and those advantages provided are for illustration. The statements in this section merely provide the background related to the present disclosure and do not constitute prior art.

### SUMMARY OF THE INVENTION

This summary is provided to introduce a selection of concepts in a simplified form that are further described below in the DESCRIPTION OF THE DISCLOSURE. This summary is not intended to identify key features of the claimed subject matter, nor is it intended to be used as an aid in determining the scope of the claimed subject matter.

In accordance with one or more embodiments of the present invention, a nasal spray solution is disclosed. The nasal spray solution comprises: an effective amount of vitamin B12; an effective amount of aloe vera extract; an effective amount of L-glutamine; an effective amount of potassium chloride; and purified water.

In accordance with one or more embodiments of the present invention, a nasal spray solution is disclosed. The nasal spray solution comprises: approximately 0.10% concentration of an active form of vitamin B12; approximately 0.50% concentration of aloe vera extract; approximately 0.20% concentration of L-glutamine; approximately 0.30% concentration of potassium chloride; approximately 1.50% concentration of ectoin; approximately 0.90% concentration of sodium chloride; approximately 0.50% concentration of xylitol; approximately 0.50% concentration of vitamin C; approximately 0.10% concentration of vitamin B6; approximately 0.30% concentration of magnesium chloride; approximately 0.20% of N-acetylcysteine; approximately 0.01% of preservative; and a remainder of the nasal spray solution being purified water.

In accordance with one or more embodiments of the present invention, a method for preparation of a nasal spray is disclosed. The method comprises the steps of: preparing a first mixture by dissolving effective amounts of ectoin, vitamin B12, vitamin C, vitamin B6, magnesium chloride, sodium chloride, and potassium chloride in an amount of purified water under constant stirring; preparing a second mixture by dissolving in a separate container effective amounts of L-glutamine, N-acetylcysteine, and xylitol in another amount of purified water; combining the first mixture with the second mixture; adding an effective amount of aloe vera extract; adding an effective amount of sodium hydroxide or hydrochloric acid to maintain a pH of the nasal spray solution between 5.5 and 6.5; adding an effective amount of benzalkonium chloride as a preservative; and adding an amount of purified water to achieve a final volume of the nasal spray solution.

### BRIEF DESCRIPTION OF THE DRAWING

The novel features believed to be characteristic of the disclosure are set forth in the appended claims. The disclosure itself, as well as a preferred mode of use, further objectives and advantages thereof, will be best understood by reference to the following detailed description of illustrative embodiments when read in conjunction with the accompanying drawing, wherein:

FIG. 1 is a flow diagram of one example of a method for preparing the nasal spray solution in accordance with one aspect of the present invention.

### DETAILED DESCRIPTION OF THE INVENTION

The description set forth below in connection with the appended drawings is intended as a description of exemplary embodiments of the disclosure and is not intended to represent the only forms in which the present disclosure may be constructed and/or utilized. The description sets forth the functions and the sequence of blocks for constructing and operating the disclosure in connection with the illustrated embodiments. It is to be understood, however, that the same or equivalent functions and sequences may be accomplished by different embodiments that are also intended to be encompassed within the spirit and scope of this disclosure.

The nasal spray solution of the present invention may be used to quickly and discretely treat common hangover symptoms. Hangover symptoms may include, but are not limited to, headache, fatigue, nausea, redness of the eyes and/or nose, thirst, raised blood pressure, congestion, and dizziness. Although the present invention is described herein as being used to relieve hangover symptoms resulting from alcohol consumption, it should be clearly understood that the nasal spray solution may be used to relieve symptoms including headache, fatigue, nausea, redness of the eyes and/or nose, thirst, raised blood pressure, congestion, and dizziness, etc. even though those symptoms may not be related to a hangover.

In its simplest form, the nasal spray solution may comprise the following active ingredients/components: Vitamin B12 (methylcobalamin), potassium chloride, L-glutamine, and aloe vera extract. Additionally, the nasal spray solution may also comprise the following: ectoin, sodium chloride, xylitol, and Vitamin C. Still further, the nasal spray solution may also comprise the following: Vitamin B6, magnesium chloride, N-acetylcysteine (NAC), preservative (e.g. benzalkonium chloride), and purified water. These components may be added to the solution individually. Alternatively, some of the components may be pre-combined prior to being added to the solution.

Vitamin B12 is an essential water-soluble vitamin that may be found in foods like fish, dairy, and meat. Vitamin B12 is needed for the development and function of several parts of the body, including the brain, nerves, and blood cells. Methylcobalamin is the active form of Vitamin B12. Vitamin B12 may be used for increasing production of red blood cells, reducing fatigue, and increasing energy and metabolism. Vitamin B12 supports energy metabolism, neurological function, and red blood cell formation.

Potassium chloride is a potassium salt that is used to treat hypokalemia (i.e. low blood potassium). Potassium helps the body's cells, kidneys, heart, nerves, and muscles function properly. Potassium chloride may be used to replenish one's body with the minerals needed to retain fluids and expel toxins. Potassium chloride helps to maintain electrolyte balance and supports proper cell function.

L-glutamine is an essential amino acid that supports the body's immune and digestive systems. L-glutamine may be used to reduce symptoms of alcohol withdrawal, such as headaches, tremors, nausea, anxiety, insomnia, or hallucinations. L-glutamine supports immune function, gut health, and muscle recovery.

Aloe vera extract is collected from an aloe vera plant. It may be used to coat the stomach lining, allowing it to reduce inflammation and balance out acidity in the gut. This may help to reduce nausea and vomiting. Aloe vera extract soothes and hydrates nasal passages and it has anti-inflammatory properties.

Ectoin is a natural amino acid compound that may be found in some types of bacteria and microorganisms. Ectoin is a natural stress-protecting molecule that helps organisms survive in extreme conditions. Ectoin helps to protect cells from environmental stress, reduces inflammation, and stabilizes cell membranes.

Sodium chloride is also known as common table salt. Sodium chloride is the primary salt in seawater and in the extracellular fluid of many multicellular organisms. Sodium chloride is essential because it absorbs and transports nutrients, maintains blood pressure, and maintains the balance of fluid in the body. Sodium chloride also hydrates and clears nasal passages.

Xylitol is a sugar alcohol that is commonly used as an artificial sweetener or sugar substitute. Xylitol occurs naturally in many fruits and vegetables and it is very soluble in water. Xylitol moisturizes nasal passages, reduces bacterial adhesion, and supports hydration.

Vitamin C is a water-soluble vitamin and antioxidant. Vitamin C is essential for the body's normal growth and development. Vitamin C helps mitigate oxidative stress, supports collagen synthesis, and immune function.

Vitamin B6 (pyridoxine hydrochloride) is a water-soluble vitamin that helps metabolize amino acids and glycogen. Vitamin B6 is involved in amino acid metabolism and neurotransmitter synthesis, and it may alleviate nausea.

Magnesium chloride is an inorganic compound. Magnesium chloride may be used to regulate blood sugar levels and blood pressure and it may be used for making protein, bone, and DNA. Magnesium chloride supports muscle function, nerve transmission, and energy production.

N-acetylcysteine (NAC) is a medication and dietary supplement that has anti-inflammatory and antioxidant properties. N-acetylcysteine is a precursor to glutathione. It helps to detoxify the body and also has mucolytic properties.

Preservatives may be used to improve the shelf life of products like medicine or food. Benzalkonium chloride is a quaternary ammonium compound that may be used as a preservative. Benzalkonium chloride prevents microbial contamination and ensures product safety.

Purified water is water that has been filtered and treated to remove impurities and contaminants in order for it to be safe for cleaning and consumption. Purified water may be used as a solvent for ensuring even distribution and administration of ingredients in a liquid solution. Purified water may also be used to hydrate nasal passages.

A nasal spray solution in accordance with the present invention may include the four main ingredients in the effective amounts identified below. In the examples below, “q.s.” for purified water should be understood to mean that as much as is enough purified water is added to make 100%. It should be clearly understood that these % concentrations are provided as examples, and the disclosure should not be limited to these examples.

### Example 1

| Component | % Concentration |
|---|---|
| Vitamin B12 (Methylcobalamin) | 0.25% |
| Aloe Vera Extract | 0.30% |
| L-Glutamine | 0.35% |
| Potassium Chloride | 0.45% |
| Purified Water | q.s. to 100% |

Another nasal spray solution in accordance with the present invention may include the following ingredients in the effective amounts identified below.

### Example 2

| Component | % Concentration |
|---|---|
| Vitamin B12 (Methylcobalamin) | 0.10-0.25% |
| Aloe Vera Extract | 0.30%-0.50% |
| L-Glutamine | 0.20-0.35% |
| Potassium Chloride | 0.30-0.45% |
| Ectoin | 1.50% |
| Sodium Chloride | 0.90% |
| Xylitol | 0.50% |
| Vitamin C | 0.50% |
| Purified Water | q.s. to 100% |

Another nasal spray solution in accordance with the present invention may include the following ingredients in the effective amounts identified below.

### Example 3

| Component | % Concentration |
|---|---|
| Vitamin B12 (Methylcobalamin) | 0.10-0.25% |
| Aloe Vera Extract | 0.30%-0.50% |
| L-Glutamine | 0.20-0.35% |
| Potassium Chloride | 0.30-0.45% |
| Ectoin | 1.50% |
| Sodium Chloride | 0.90% |
| Xylitol | 0.50% |
| Vitamin C | 0.50% |
| Vitamin B6 (Pyridoxine Hydrochloride) | 0.10% |
| Magnesium Chloride | 0.30% |
| N-Acetylcysteine (NAC) | 0.20% |
| Preservative (Benzalkonium Chloride) | 0.01% |
| Purified Water | q.s. to 100% |

Another nasal spray solution in accordance with the present invention may include the following ingredients in the effective amounts identified below.

### Example 4

| Component | % Concentration |
|---|---|
| Vitamin B12 (Methylcobalamin) | 0.10% |
| Aloe Vera Extract | 0.50% |
| L-Glutamine | 0.20% |
| Potassium Chloride | 0.30% |
| Ectoin | 1.50% |
| Sodium Chloride | 0.90% |
| Xylitol | 0.50% |
| Vitamin C | 0.50% |
| Vitamin B6 (Pyridoxine Hydrochloride) | 0.10% |
| Magnesium Chloride | 0.30% |
| N-Acetylcysteine (NAC) | 0.20% |
| Preservative (Benzalkonium Chloride) | 0.01% |
| Purified Water | q.s. to 100% |

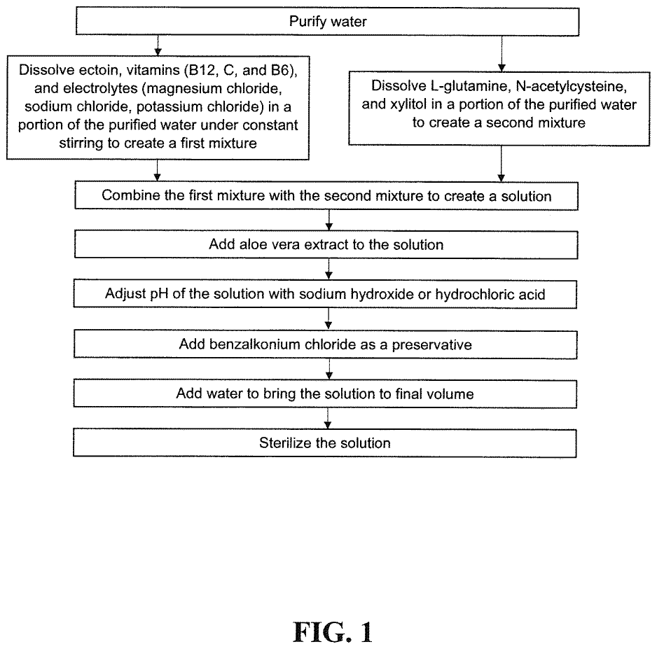

When preparing the nasal spray solution, pH balance, osmolarity, stability, and absorption should be considered. Referring to FIG. 1, the nasal spray solution of Example 4 above may be prepared according to the following process. Water should be purified in a clean and sterile environment. Ectoin, vitamins (i.e. Vitamin B12, Vitamin C, and Vitamin B6), and electrolytes (magnesium chloride, sodium chloride, and potassium chloride) may be dissolved in a portion of the water under constant stirring to create a first mixture.

In a separate container, L-glutamine, N-acetylcysteine, and xylitol may be dissolved in another portion of the water to make a second mixture. The two mixtures may then be combined to form one solution. Aloe vera extract may be added to the solution. The mixture should be stirred thoroughly to ensure homogeneity. The first mixture and the second mixture should be prepared at room temperature. However, if the ambient temperature is too low, the first mixture and the second mixture may be mixed at around 77-80 degrees Fahrenheit.

The pH may be adjusted to between approximately 5.5 and 6.5 by using either sodium hydroxide or hydrochloric acid. Benzalkonium chloride may then be added as a preservative. The solution is then brought to final volume with additional purified water and mixed well.

The entire solution may be passed through a 0.22-micron filter for sterilization. Finally, the filtered solution is aseptically filled into pre-sterilized nasal spray bottles.

Throughout the entire process, strict quality control measures should be taken, including checks for pH, osmolality, and microbial contamination. Stability testing should also be conducted to ensure the nasal spray solution maintains its quality throughout its shelf life. Regarding pH balance, the pH should be close to that of nasal mucosa (approximately 6.3 to 6.8) in order to prevent irritation. The osmolarity of the nasal spray solution should match the osmolarity of bodily fluids to ensure comfort and proper absorption. Regarding stability, the nasal spray solution should be formulated to maintain ingredient stability and efficacy over the product's shelf life. The nasal spray solution should also be formulated to optimize the absorption of active ingredients through nasal mucosa, potentially using absorption enhancers.

The foregoing description is provided to enable any person skilled in the relevant art to practice the various embodiments described herein. Various modifications to these embodiments will be readily apparent to those skilled in the relevant art and generic principles defined herein may be applied to other embodiments. Thus, the claims are not intended to be limited to the embodiments shown and described herein, but are to be accorded the full scope consistent with the language of the claims, wherein reference to an element in the singular is not intended to mean “one and only one” unless specifically stated, but rather “one or more.” All structural and functional equivalents to the elements of the various embodiments described throughout this disclosure that are known or later come to be known to those of ordinary skill in the relevant art are expressly incorporated herein by reference and intended to be encompassed by the claims. Moreover, nothing disclosed herein is intended to be dedicated to the public regardless of whether such disclosure is explicitly recited in the claims.

What is claimed is:

1. A nasal spray solution comprising:
an effective amount of vitamin B12;
an effective amount of aloe vera extract;
an effective amount of L-glutamine;
an effective amount of potassium chloride; and
purified water.

2. The nasal spray solution of claim 1 comprising:
approximately 0.25% concentration of vitamin B12;
approximately 0.30% concentration of aloe vera extract;
approximately 0.35% concentration of L-glutamine;
approximately 0.45% concentration of potassium chloride; and
a remainder of the nasal spray solution being purified water.

3. The nasal spray solution of claim 1 further comprising:
an effective amount of ectoin;
an effective amount of sodium chloride;
an effective amount of xylitol; and
an effective amount of vitamin C.

4. The nasal spray solution of claim 3 comprising:
between approximately 0.10-0.25% concentration of vitamin B12;
between approximately 0.30-0.50% concentration of aloe vera extract;
between approximately 0.20-0.35% concentration of L-glutamine;
between approximately 0.30-0.45% concentration of potassium chloride;
approximately 1.50% concentration of ectoin;
approximately 0.90% concentration of sodium chloride;
approximately 0.50% concentration of xylitol;
approximately 0.50% concentration of vitamin C; and
a remainder of the nasal spray solution being purified water.

5. The nasal spray solution of claim 3 further comprising:
an effective amount of vitamin B6;
an effective amount of magnesium chloride;
an effective amount of N-acetylcysteine; and
an effective amount of preservative.

6. The nasal spray solution of claim 5 comprising:
between approximately 0.10-0.25% concentration of vitamin B12;
between approximately 0.30-0.50% concentration of aloe vera extract;
between approximately 0.20-0.35% concentration of L-glutamine;
between approximately 0.30-0.45% concentration of potassium chloride;
approximately 1.50% concentration of ectoin;
approximately 0.90% concentration of sodium chloride;
approximately 0.50% concentration of xylitol;
approximately 0.50% concentration of vitamin C;
approximately 0.10% concentration of vitamin B6;
approximately 0.30% concentration of magnesium chloride;
approximately 0.20% of N-acetylcysteine;
approximately 0.01% of preservative; and
a remainder of the nasal spray solution being purified water.

7. The nasal spray solution of claim 6 comprising:
approximately 0.10% concentration of vitamin B12;
approximately 0.50% concentration of aloe vera extract;
approximately 0.20% concentration of L-glutamine; and
approximately 0.30% concentration of potassium chloride.

8. The nasal spray solution of claim 1 wherein the vitamin B12 is methylcobalamin.

9. The nasal spray solution of claim 5 wherein the vitamin B6 is pyridoxine hydrochloride.

10. The nasal spray solution of claim 5 wherein the preservative is benzalkonium chloride.

11. The nasal spray solution of claim 1 wherein the pH of the nasal spray solution is between 5.5 and 6.5.

12. The nasal spray solution of claim 11 further comprising an effective amount of sodium hydroxide or hydrochloric acid to affect the pH of the nasal spray solution.

13. A nasal spray solution comprising:
approximately 0.10% concentration of an active form of vitamin B12;
approximately 0.50% concentration of aloe vera extract;
approximately 0.20% concentration of L-glutamine;
approximately 0.30% concentration of potassium chloride;
approximately 1.50% concentration of ectoin;
approximately 0.90% concentration of sodium chloride;
approximately 0.50% concentration of xylitol;
approximately 0.50% concentration of vitamin C;
approximately 0.10% concentration of vitamin B6;
approximately 0.30% concentration of magnesium chloride;
approximately 0.20% of N-acetylcysteine;
approximately 0.01% of preservative; and
a remainder of the nasal spray solution being purified water.

14. The nasal spray solution of claim 12 wherein the active form of vitamin B12 is methylcobalamin.

15. The nasal spray solution of claim 13 wherein the vitamin B6 is pyridoxine hydrochloride.

16. The nasal spray solution of claim 13 wherein the preservative is benzalkonium chloride.

17. The nasal spray solution of claim 13 further comprising an effective amount of sodium hydroxide or hydrochloric acid to maintain a pH of the nasal spray solution between 5.5 and 6.5.

18. A method for preparing a nasal spray solution comprising the steps of:
preparing a first mixture by dissolving effective amounts of ectoin, vitamin B12, vitamin C, vitamin B6, magnesium chloride, sodium chloride, and potassium chloride in an amount of purified water under constant stirring;
preparing a second mixture by dissolving in a separate container effective amounts of L-glutamine, N-acetylcysteine, and xylitol in another amount of purified water;
combining the first mixture with the second mixture;
adding an effective amount of aloe vera extract;
adding an effective amount of sodium hydroxide or hydrochloric acid to maintain a pH of the nasal spray solution between 5.5 and 6.5;
adding an effective amount of benzalkonium chloride as a preservative; and
adding an amount of purified water to achieve a final volume of the nasal spray solution.

19. The method of claim 18 wherein the first mixture comprises:
approximately 0.10% concentration of an active form of vitamin B12;
approximately 0.30% concentration of potassium chloride;
approximately 1.50% concentration of ectoin;
approximately 0.90% concentration of sodium chloride;
approximately 0.50% concentration of vitamin C;
approximately 0.10% concentration of vitamin B6; and
approximately 0.30% concentration of magnesium chloride.

20. The method of claim 18 wherein the second mixture comprises:
approximately 0.20% concentration of L-glutamine;
approximately 0.50% concentration of xylitol; and
approximately 0.20% of N-acetylcysteine.
